(19) United States
(12) Patent Application Publication
(10) Pub. No.: US 2020/0319131 A1
(43) Pub. Date: Oct. 8, 2020

(54) ELECTROCHEMICAL METHOD TO DETECT ARSENIC (III) IONS IN WATER USING NANOSTRUCTURED COLLOIDAL METALS

(71) Applicant: Wisconsin Alumni Research Foundation, Madison, WI (US)

(72) Inventors: Sundaram Gunasekaran, Madison, WI (US); Batul Kachwala, Middleton, WI (US); Anu Prathap Mylamparambil Udayan, Madison, WI (US)

(21) Appl. No.: 16/835,923

(22) Filed: Mar. 31, 2020

Related U.S. Application Data
(60) Provisional application No. 62/828,596, filed on Apr. 3, 2019.

(51) Int. Cl. G01N 27/30; G01N 33/18; G01N 27/00

(52) U.S. Cl. CPC G01N 27/30; G01N 27/002; G01N 33/1813

(57) ABSTRACT

Monodispersed colloidal gold nanoparticles (AuNPs) were synthesized by an easy, cost-effective, and eco-friendly synthesis route. The resulting AuNPs exhibited excellent electroanalytical ability to simultaneously detect toxic As(III) and As(V). The limit of quantification (LOQ) toward As(III) was 0.075 ppb (1 nM), which is well below the guideline value approved by the United States Environmental Protection Agency (US EPA) and the World Health Organization (WHO). Under the optimal conditions, a linear response in the concentration range of from about 0.075 ppb to about 0.03 ppm (1 nM-400 nM) was observed. The method is useful to detect arsenic contamination of water intended for human and animal consumption.

### CROSS-REFERENCE TO RELATED APPLICATIONS

Priority is hereby claimed to provisional application Ser. No. 62/828,596, filed Apr. 3, 2019, which is incorporated herein by reference.

### BACKGROUND

The last two decades have witnessed a growing issue pertaining to arsenic poisoning worldwide. (Acharyya et al., Arsenic Poisoning in the Ganges Delta. Nature 1999, 401 (6753), 545. Nickson et al., Arsenic Poisoning of Bangladesh Groundwater. Nature 1998, 395 (6700), 338. Cullen and Reimer, Arsenic Speciation in the Environment. Chem. Rev. 1989, 89 (4), 713-764.) Contamination of drinking water by arsenic is found in several parts of the globe, threatening the health of roughly 140 million people. (Islam et al., Role of Metal-Reducing Bacteria in Arsenic Release from Bengal Delta Sediments. Nature 2004, 430 (6995), 68-71.) Although arsenic exists in different types in nature, in groundwater it exists primarily in two inorganic forms: pentavalent arsenate (As(V)), and trivalent arsenite (As(III)). Depending on pH, there are different forms of As. At lower redox potentials and under the neutral and mildly alkaline conditions (pH<9), the trivalent arsenic species (H₃AsO₃, H₂AsO₃⁻, HAsO₃²⁻, AsO₃³⁻) become stable, whereas at higher redox potentials arsenic is stabilized as a series of pentavalent oxy-arsenic species such as H₃AsO₄, H₂AsO₄⁻, HAsO₄²⁻, and AsO₄³⁻. As(III) has proven to be much more harmful to human health, with toxicity at least 60 times higher than that of As(V) and other organic arsenic types. (Zen et al., Flow Injection Analysis of an Ultratrace Amount of Arsenite Using a Prussian Blue-Modified Screen-Printed Electrode. Anal. Chem. 2003, 75 (21), 6017-6022.) Persistent exposure to As(III) above the World Health Organization and U.S. Environmental Protection Agency threshold value of 10 ppb causes a number of diseases, including skin damage, issues with the circulatory system, and several different types of cancers, including those of the skin, the lungs, the bladder and the prostate. Lage et al., Arsenic Ecotoxicology and Innate Immunity. Integr Comp Biol 2006, 46 (6), 1040-1054.) On the south-western coast of Taiwan, local artesian well waters having As(III) concentrations of 500±130 μg L⁻¹ and As(V) concentrations of 200±110 μL⁻¹ have been identified. These high concentrations of arsenic ions contribute to the high incidence of Blackfoot disease (BFD) and urinary cancer in the area. (Li et al., The Oxidative Transformation of Sodium Arsenite at the Interface of α-MnO2 and Water. Journal of Hazardous Materials 2010, 173 (1), 675-681. Tremiliosi-Filho et al., Growth of Surface Oxides on Gold Electrodes under Well-Defined Potential, Time and Temperature Conditions. Journal of Electroanalytical Chemistry 2005, 578 (1), 1-8. Sitko et al., Green Approach for Ultratrace Determination of Divalent Metal Ions and Arsenic Species Using Total-Reflection X-Ray Fluorescence Spectrometry and Mercapto-Modified Graphene Oxide Nanosheets as a Novel Adsorbent. Anal. Chem. 2015, 87 (6), 3535-3542.)

As a result of the public health hazard of arsenic in drinking water, several analytical strategies have been developed to measure arsenic concentration in water. (See Li et al., Tremiliosi-Filho et al., and Sitko et al. supra.) These analytical methods usually involve laboratory-based instruments such as surface enhanced Raman spectroscopy (SERS), atomic absorption spectroscopy (AAS), flame atomic absorption spectroscopy (FAAS), graphite furnace atomic absorption spectroscopy (GFAAS), and inductively coupled plasma-mass spectroscopy (ICP-MS). (B'Hymer and Caruso, Arsenic and Its Speciation Analysis Using High-Performance Liquid Chromatography and Inductively Coupled Plasma Mass Spectrometry. Journal of Chromatography A 2004, 1045 (1), 1-13. Liu et al., Enzyme-Assisted Extraction and Liquid Chromatography Mass Spectrometry for the Determination of Arsenic Species in Chicken Meat. Analytica Chimica Acta 2015, 888, 1-9. Musil et al. Speciation without Chromatography Using Selective Hydride Generation: Inorganic Arsenic in Rice and Samples of Marine Origin. Anal. Chem. 2014, 86 (2), 993-999.) But these methods are not simple, are not cheap, and cannot be implemented in the field. Consequently, there has been a long-felt and unmet need for a simple, rapid, robust, and field-implementable analytical method for determining arsenic concentration in water. Aptamers have been considered as appealing tools in heavy metal monitoring present in the environment. The arsenic-binding DNA aptamer, Ars-3, has the highest affinity for As(III). Surface-enhanced Raman spectroscopy (SERS) based on Ars-3 can simplify the detection procedure and improve the selectivity for As(III) detection, but even so the method requires expensive Raman equipment. (Kim et al., Arsenic Removal from Vietnamese Groundwater Using the Arsenic-Binding DNA Aptamer. Environ. Sci. Technol. 2009, 43 (24), 9335-9340. Wu et al., Cationic Polymers and Aptamers Mediated Aggregation of Gold Nanoparticles for the Colorimetric Detection of Arsenic(III) in Aqueous Solution. Chem. Commun. 2012, 48 (37), 4459-4461.

Electrochemical approaches are also described in the literature as an alternative for the conventional analytical methods. The electrochemical strategies described in the prior art offer several advantages, notably their low cost and user-friendly format. Generally, these methods use cathodic and anodic stripping voltammetry (CSV/ASV) techniques. Here, the arsenic-containing solution is pre-concentrated, followed by deposition of the arsenic ions at a ligand- or biomolecule-modified electrode, followed by stripping of these arsenic species from the electrode. The sensor electrode then forms a selective complex with arsenic ions, whereby their concentration is determined. Electrochemical biosensing based on the inhibitory function of some of the enzymes such as acetylcholinesterase, acid phosphatase, polyphenol oxidase or laccase with arsenic, and also an electro-catalytic approach utilizing redox-mediated oxidation/reduction of the arsenic ion, have been reported in the recent literature. However, the usefulness of these prior art methods is limited due to interference from various other, commonly encountered metals such as copper (Cu), mercury (Hg), selenium (Se)—a problem that has yet to be addressed because the stripping peak of these metals are seen at a comparable potential to that of As. For example, in the CSV evaluation, As(III) ions were pre-concentrated at a negative potential (−0.5 V versus silver/silver chloride (Ag/AgCl)) in the presence of Cu(II) or Se(IV) as a CuₓAs_{y} intermetallic compound on a mercury (Hg) electrode prior to the stripping analysis. Hence, an analytical challenge for determining As(III) concentration is a detection mechanism that is effective down to parts-per-billion (ppb) or sub-ppb levels of As(III), without interference from other metal ions commonly encountered in water destined for human and animal consumption. Anu et al., Ultrasensitive Electrochemical Immunoassay for Melanoma Cells Using Mesoporous Polyaniline. Chemical Communications 2018, 54 (7), 710-714. Anu et al., Electrochemical Sensor Platforms Based on Nanostructured Metal Oxides, and Zeolite-Based Materials. The Chemical Record 2018, 0 (0). Liu et al., Voltammetric Determination of Inorganic Arsenic. TrAC Trends in Analytical Chemistry 2014, 60, 25-35. Forsberg et al., Determination of Arsenic by Anodic Stripping Voltammetry and Differential Pulse Anodic Stripping Voltammetry. Anal. Chem. 1975, 47 (9), 1586-1592. Stoytcheva et al., Electrochemical Approach in Studying the Inhibition of Acetylcholinesterase by Arsenate(III): Analytical Characterisation and Application for Arsenic Determination. Analytica Chimica Acta 1998, 364 (1), 195-201. Cosnier et al., Specific Determination of As(V) by an Acid Phosphatase-Polyphenol Oxidase Biosensor. Anal. Chem. 2006, 78 (14), 4985-4989. Wang et al., Inhibition by Arsenite/Arsenate: Determination of Inhibition Mechanism and Preliminary Application to a Self-Powered Biosensor. Anal. Chem. 2016, 88 (6), 3243-3248. Ferreira and Barros, Determination of As(III) and Arsenic(V) in Natural Waters by Cathodic Stripping Voltammetry at a Hanging Mercury Drop Electrode. Analytica Chimica Acta 2002, 459 (1), 151-159.

### SUMMARY

Disclosed herein is a method to detect and to quantify arsenic in water (generally), and arsenic(III) ions in water (specifically). As described below, approximately 15±3 nm-sized colloidal AuNPs were synthesized by a facile reduction method. Impressively, the AuNPs exhibited rapid detection of As(III). The linear range of detection was from approximately 0.075 ppb (1 nM) to approximately 0.03 ppm (400 nM) with a lower limit of quantitation (LOQ) of about 0.075 ppb (about 1 nM) (R²=0.9838, n=5). AuNPs-modified electrodes demonstrated high sensitivity, repeatability, reproducibility and long-term stability. Without being limited to any specific mechanism or phenomenon, an electrochemical reaction mechanism for As(III) oxidation is proposed. The AuNPs-modified electrode disclosed herein was successfully employed to quantify the concentration of As(III) ions in authentic tap water samples.

Thus, disclosed herein is a method of detecting arsenic in a sample. The method comprises contacting the sample with a working electrode and a counter electrode in the presence of an electrolyte. The working electrode comprises a nanoparticulate noble metal having a mean particle size of from about 10 nm to about 20 nm adhered to a supporting electrode material. The working electrode is dimensioned and configured to generate an electrochemical response that is proportional to the concentration of arsenic in the sample. A potential is applied across the electrodes. This generates an electrochemical response at the working electrode which is proportional to the amount and/or concentration of arsenic in the sample tested.

In the preferred version of the method, the working electrode comprises nanoparticulate gold. The nanoparticulate gold most preferably has a mean particle size of from about about 12 nm to about 18 nm. The sample tested is typically a liquid sample, and most commonly an aqueous sample.

The potential applied to the electrodes can be a fixed voltage or a ramped, varying voltage.

In the most preferred version, the method comprises contacting an aqueous sample with a working electrode and a counter electrode in the presence of an electrolyte. Here, the working electrode comprises a nanoparticulate gold having a mean particle size of from about 12 nm to about 18 nm adhered to a supporting electrode material. Again, the working electrode is dimensioned and configured to generate an electrochemical response that is proportional to the concentration of arsenic in the sample. A varying potential (of any desired voltage profile) is applied across the working electrode and the counter electrode relative to a reference electrode. The voltammetric response of the working electrode relative to the potential applied is then determined. The voltammetric response determined is proportional to the concentration of arsenic in the aqueous sample.

Also disclosed herein is the electrochemical detector used in the method. The electrochemical detector comprises, in combination, a reference electrode, a counter electrode, and a working electrode comprising noble metal nanoparticles having a mean particle size of from about 10 nm to about 20 nm, adhered to a supporting electrode material. In a preferred version, the reference electrode comprises silver and silver chloride, the counter electrode comprises carbon, and the supporting electrode material of the working electrode comprises carbon. All of the various electrodes may be fabricated by screen printing. The preferred noble metal for the working material is gold.

Also disclose herein is a method to make the electrochemical detector. The detector is made via a process comprising screen-printing onto a substrate a reference electrode, a counter electrode, and a working electrode; and drop casting noble metal nanoparticles onto the working electrode. The method may comprise drop casting noble metal nanoparticles, preferably gold nanoparticles, having a mean particle size of from about 10 nm to about 20 nm onto the working electrode, and more preferably still about 12 nm to about 18 nm.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The indefinite articles “a” and “an” are explicitly defined herein to mean “one or more” or “at least one” unless explicitly stated to the contrary.

All combinations of method or process steps as used herein can be performed in any order, unless otherwise specified or clearly implied to the contrary by the context in which the referenced combination is made.

The apparatus and methods described herein can comprise, consist of, or consist essentially of the essential elements and limitations disclosed herein, as well as any additional or optional ingredients, components, or limitations described herein or otherwise useful in electrochemical detection of ions in solution.

### BRIEF DESCRIPTION OF THE DRAWINGS

FIG. 1 is a schematic diagram depicting synthesis of quasi-hexagonal gold nanoparticles (AuNPs).

FIG. 2A is an XPS spectrum of the Au 4f level of AuNPs.

FIG. 2B is a HRTEM image of Au.

FIG. 2C is a particle size histogram for AuNPs generated by DLS.

FIG. 2D is a UV-Vis spectrum of as-synthesized AuNPs. The inset is a picture of AuNPs in aqueous solution.

FIG. 3A is a cyclic voltammogram of a AuNPs/SPE (screen-printed electrode) in 1.0 M H₂SO₄ and PBS buffer (pH=6.4) at a scan rate of 50 mV/s.

FIG. 3B is a cyclic voltammogram of a AuNPs/SPE in 1.0 M NaOH and PBS buffer (pH=6.4) at a scan rate of 50 mV/s.

FIG. 4A is a differential pulse anodic stripping voltammogram (DPASV) of a AuNPs/SPE in PBS buffer at pHs ranging of 6.4 to 10.

FIG. 4B is a graph showing the dependence of the peak potentials as a function of pH for the AuNPs/SPE electrode analysed in FIG. 4A.

FIG. 4C is a DPASV with varying concentrations of AS(III) and As(V) ions obtained with the AuNPs/SPE (1 and 50 nM).

FIG. 5A is a DPASV with varying concentrations of As(III) at a AuNPs/SPE (pH=6.4).

FIG. 5B is an enlarged view of FIG. 5A at low concentrations of As(III) (0 nM, 1 nM, and 20 nM As(III)).

FIG. 5C is the calibration curve generated from the data shown in FIGS. 5A and 5B.

FIG. 6A is a DPASV of AuNPs/SPE in PBS buffer (pH=6.4) showing the simultaneous detection of Cd(II), Cu(II), and Hg(II) all at 0, 100, 200, 400, 600, and 1000 nM and As(III) at 0, 10, 50 100, 150, and 200 nM.

FIG. 6B is a DPASV of AuNPs/SPE in PBS buffer (pH=6.4) showing the simultaneous detection of a binary mixture of Cu(II) and As(III).

FIG. 6C is a DPASV showing simultaneous detection of a binary mixture of Cu(II) and As(III) at different pHs ranging of pH 6.4 to pH 10.

FIG. 6D is a DPASV of AuNPs/SPE in PBS buffer (pH=6.4) showing the simultaneous detection of Pb(II) at 200, 400, and 1000 nM and As(III) at 200 nM.

FIG. 7A is a DPASV with varying concentrations of As(III) at a AuNPs/SPE in tap water (pH=7.8).

FIG. 7B is the calibration curve generated from the data shown in FIG. 7A.

FIG. 8A depicts a high-resolution XPS spectrum for all elements for As(III) detection at a AuNPs/SPE.

FIG. 8B is a magnified view of the XPS spectrum of FIG. 8A for Au 4f electrons.

FIG. 8C is a magnified view of the XPS spectrum of FIG. 8A for As 3d electrons.

FIG. 8D is a magnified view of the XPS spectrum of FIG. 8A for O 1s electrons.

FIG. 8E is a magnified view of the XPS spectrum of FIG. 8A for C 1s electrons.

FIG. 9 is a schematic diagram of electrochemical sensing of As(III) on AuNPs/SPE according to the present method.

### DETAILED DESCRIPTION

### ABBREVIATIONS AND DEFINITIONS

AAS=atomic absorption spectroscopy.

ASV=anodic stripping voltammetry.

AuNPs=colloidal gold nanoparticles.

CSV=cathodic stripping voltammetry.

CV=cyclic voltammetry.

DI=deionized.

DLS=dynamic light scattering.

DPASV=differential pulse anodic stripping voltammetry.

DPV=differential pulse voltammetry.

FAAS=flame atomic absorption spectroscopy.

GFAAS=graphite furnace atomic absorption spectroscopy.

HRTEM=high-resolution transmission electron microscopy.

ICPMS=inductively coupled plasma mass spectroscopy.

LOD=limit of detection.

LOQ=limit of quantitation.

PBS=phosphate-buffered solution.

SERS=surface-enhanced Raman spectroscopy.

SPE=screen-printed electrode.

SPR=surface plasmon resonance.

“Noble metals” are defined herein (in order of increasing atomic number) as copper (Cu), ruthenium (Ru), rhodium (Rh), palladium (Pd), silver (Ag), rhenium (Re), osmium (Os), iridium (Ir), platinum (Pt), gold (Au), and mercury (Hg).

“Transition metals” are defined herein as those elements within groups 3 to 12 on the periodic table, as well as the f-block lanthanide and actinide series of elements. The noble metals are a sub-set within the transition metals.

XPS=X-ray photoelectron spectroscopy.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. Unless specifically stated to the contrary, the indefinite articles “a” and “an” mean “one or more.” The phrase “one or more” is readily understood by one of skill in the art, particularly when read in context of its usage. For example, “one or more” substituents on a phenyl ring designates one to five substituents.

All combinations of method or process steps as used herein can be performed in any order, unless otherwise specified or clearly implied to the contrary by the context in which the referenced combination is made.

The steps of the disclosed method can comprise, consist of, or consist essentially of the essential elements and limitations of the method disclosed herein, as well as any additional or optional ingredients, components, or limitations described herein or otherwise useful in the electrochemical detection of ions in solution.

The term “contacting” refers to the act of touching, making contact, or of bringing to immediate or close proximity, including at the molecular level, for example, to bring about a chemical reaction, or a physical change, e.g., in a solution or in a reaction mixture or in a heterogeneous electrochemical interaction.

An “effective amount” refers to an amount of a chemical or reagent effective to facilitate a chemical reaction between two or more reaction components, and/or to bring about a recited effect. Thus, an “effective amount” generally means an amount that brings about the desired effect.

The term “solvent” refers to any liquid that can dissolve an element, molecule, compound, etc. to form a solution. Solvents include water and various organic solvents, such as hydrocarbon solvents, for example, alkanes and aryl solvents, as well as halo-alkane solvents. Examples include hexanes, benzene, toluene, xylenes, chloroform, methylene chloride, dichloroethane, and alcoholic solvents such as methanol, ethanol, propanol, isopropanol, and linear or branched (sec or tert) butanol, and the like. Aprotic solvents that can be used in the method include, but are not limited to perfluorohexane, α,α,α-trifluorotoluene, pentane, hexane, cyclohexane, methylcyclohexane, decalin, dioxane, carbon tetrachloride, freon-11, benzene, toluene, triethyl amine, carbon disulfide, diisopropyl ether, diethyl ether, t-butyl methyl ether (MTBE), chloroform, ethyl acetate, 1,2-dimethoxyethane (glyme), 2-methoxyethyl ether (diglyme), tetrahydrofuran (THF), methylene chloride, pyridine, 2-butanone (MEK), acetone, hexamethylphosphoramide, N-methylpyrrolidinone (NMP), nitromethane, dimethylformamide (DMF), acetonitrile, sulfolane, dimethyl sulfoxide (DMSO), propylene carbonate, and the like.

Materials: SPEs (TE100) were purchased from CH Instruments, Inc. (Austin, Tex., USA). Hydrogen tetrachloroaurate(III) trihydrate (HAuCl₄.3H₂O), sodium citrate, hydrochloric acid (37%), sodium chloride, sodium sulfate, silver nitrate, and sodium hydroxide, were supplied by ACROS Organics (Geel, Belgium; a wholly owned subsidiary of Thermo Fisher Scientific, Waltham, Mass.). Arsenic trioxide (As₂O₃) was purchased from Sigma-Aldrich, now Millipore Sigma (St. Louis, Mo., USA, a wholly owned subsidiary of Merck KGaA, Darmstadt, Germany). Arsenic trioxide (As₂O₃) solubility in water is 20 g/L at 25° C. The stock solution of As(III) was prepared by dissolving reagent grade As₂O₃ in deionized (DI) water. All other reagents were obtained either from Millipore Sigma or Thermo Fisher Scientific, in the highest grade available, and used without further purification. All solutions were prepared using deionized (DI) water with a resistivity of 18.2 MΩ·cm at room temperature. (The DI water was generated using an Ultrapure Water System from EMD Millipore, Burlington, Mass., USA). To produce a standard curve for As(III), a stock solution of 20 mM concentration was made using As₂O₃ in 1 mL of DI H₂O. Different concentrations (0.001 mM, 0.01 mM, and 0.1 mM) of As(III) solutions were made by diluting the primary stock solution (20 mM) using DI water.

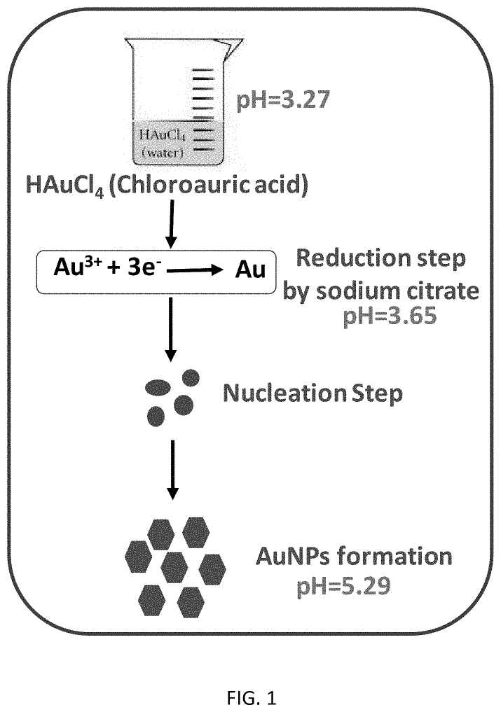

Preparation of AuNPs: Hexagonal AuNPs were synthesized according to the reported literature, using a slight modification of the Turkevich method.²³ Two (2) mL of 10 mM HAuCl₄.3H₂O was added to 18 mL DI H₂O under constant stirring and the solution was brought to a boil. A further 2 mL of 1% sodium citrate was added to the boiling HAuCl₄ with stirring in a stoppered Erlenmeyer flask. The solution turned dark brown within about 10 seconds. A final color change to burgundy occurred roughly 60 seconds later. The solution was then cooled to room temperature. The AuNPs fabrication process used herein is shown schematically in FIG. 1. The AuNPs fabrication process itself is not critical to the operation of the method or SPE disclosed herein. AuNPs made by distinct fabrication methods may also be used. The prepared solution containing AuNPs was stored under refrigerated (4° C.) and dark conditions before and after use.

Instrumentation: The X-ray photoelectron spectroscopy (XPS) was done in an XPS spectrometer to analyze the surface chemical composition and elemental distribution (Thermo Scientific K Alpha instrument). Transmission electron microscopy (TEM) images were obtained with JEOL JEM-2100F. UV-vis absorption spectra were recorded on Lambda 25-model spectrophotometer (PerkinElmer, Waltham, Mass., USA) at room temperature. Dynamic light scattering (DLS) measurements were carried out using a Nano Particle Analyzer (NanoBrook 90Plus, Brookhaven Instruments). (For background information on determining mean particle size of nanoparticles using DLS, see, for example, the NIST publication “Measuring the Size of Nanoparticles in Aqueous Media Using Batch-Mode Dynamic Light Scattering,” published Feb. 15, 2020, by the U.S. National Institute of Standards and Technology.) Electrochemical experiments were performed using CHI-660D electrochemical workstation (CH Instruments, Inc.).

Electrochemical measurements: Electrochemical experiments were performed using the CHI-660D electrochemical workstation and disposable screen-printed electrodes (SPE, also from CH Instruments, Inc.). The electrode pattern included a 3-mm diameter carbon working electrode with a geometrical surface area of 0.192 cm², a carbon counter-electrode, and a silver/silver chloride reference electrode. Electrochemical studies were carried out in the presence of 0.1 M PBS. A micro-pipette (Eppendorf Research Plus-brand, Eppendorf North America, Hauppage, N.Y. USA) was utilized to inject the analyte solution into the PBS.

Screen-printed electrodes (SPEs) are conventional articles of commerce and will be described only briefly herein. A conventional SPE comprises a suitably stiff, substrate material, typically a non-conductive polymer such as polyethylene terephthalate. In a three-electrode configuration, conductive leads for the working electrode, the counter electrode, and the reference electrode are deposited onto the substrate. The leads are typically made of silver. The electrode material for each of the three electrodes is then screen-printed onto the substrate, in operational connection with the respective lead for each of the electrodes. Thus, for example, a carbon working electrode is printed onto the substrate disk (using conductive carbon-containing ink) such that is operationally connected to the silver lead for the working electrode. The reference electrode and the counter electrode are formed in the same fashion and operationally connected to their respective leads. The method yields very robust electrode arrangements, in a huge variety of electrode materials, shapes, and sizes, and at very competitive prices. Unmodified SPE's can be obtained from a host of national and international suppliers, including CH Instruments, Inc., Metrohm DropSens (Austurias, Spain), Gamry Instruments (Warminster, Pa., USA), Pine Research (Durham, N.C., USA), and many others.

Electrode fabrication: The SPE working surface of the electrode was fabricated with AuNPs. The colloidal AuNPs (10 μL) was drop casted on the electrode and were allowed to air dry at room temperature.

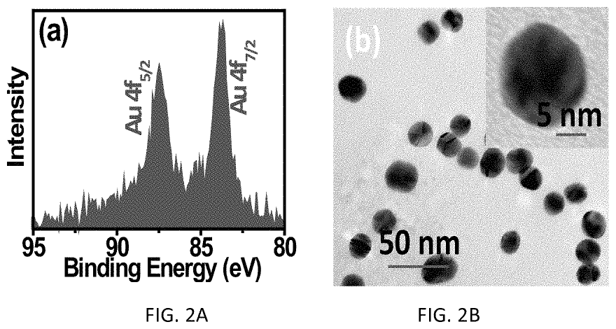

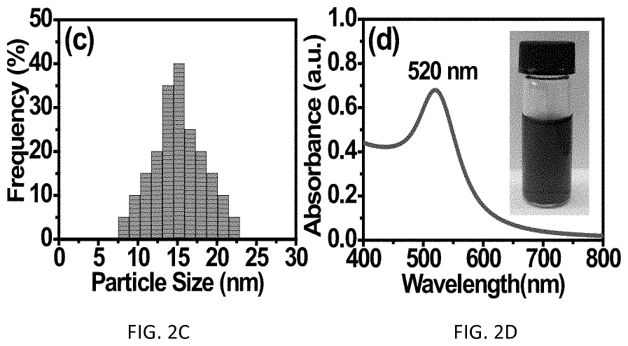

Characterization of AuNPs: X-ray photoelectron spectroscopy (XPS) was utilized to establish the composition and chemical environment of the elements present in the material. FIG. 2A is the high-resolution XPS spectrum of the Au 4f core level of the sample prepared. Two distinct lines separated by 3.78 eV were observed, namely the Au 4f_{5/2} and Au 4f_{7/2} lines, which is due to the spin-orbit splitting of the Au 4f level (FIG. 2A). (Moulder, J. F. Handbook of X-Ray Photoelectron Spectroscopy: A Reference Book of Standard Spectra for Identification and Interpretation of XPS Data; Physical Electronics Division, Perkin-Elmer Corporation, 1992.) The positions of these lines, approximated after the correction due to charge accumulation, were 87.45 eV and 83.67 eV, respectively. The peak at 83.67 eV represents Au 4f_{7/2} component, and peak at 87.45 eV corresponds to the Au 4f_{5/2} components. Carlini et al., Comparison between Silver and Gold Nanoparticles Stabilized with Negatively Charged Hydrophilic Thiols: SR-XPS and SERS as Probes for Structural Differences and Similarities. Colloids and Surfaces A: Physicochemical and Engineering Aspects 2017, 532, 183-188. Smirnov et al., Using X-Ray Photoelectron Spectroscopy To Evaluate Size of Metal Nanoparticles in the Model Au/C Samples. J. Phys. Chem. C 2016, 120 (19), 10419-10426. XPS information clearly showed the existence of Au⁰ at 83.67 eV, while other valences of gold were not seen by this method. It might be the content of gold ions was too low to be detected. (See Carlini et al., supra.)The shape and size of the AuNPs were determined by high-resolution transmission electron microscopy (TEM). A panoramic TEM image of the sample reveals ≈15 to 18 nm crystallites in a quasi-hexagonal symmetry with smaller particles exhibiting more regular shape and better dispersity. See FIG. 2B. DLS measurements of the hydrodynamic diameter of the nanoparticles agree with the particle size data obtained from the TEM measurements. The hydrodynamic diameter obtained is ˜15 nm. See FIG. 2C. UV-vis spectra of the AuNPs solutions showed plasmon resonance at 520 nm, which is characteristic of small (<15 nm) AuNPs. See FIG. 2D. (Zuber et al., Detection of Gold Nanoparticles with Different Sizes Using Absorption and Fluorescence Based Method. Sensors and Actuators B: Chemical 2016, 227, 117-127. Haiss et al., Determination of Size and Concentration of Gold Nanoparticles from UV-Vis Spectra. Anal. Chem. 2007, 79 (11), 4215-4221.

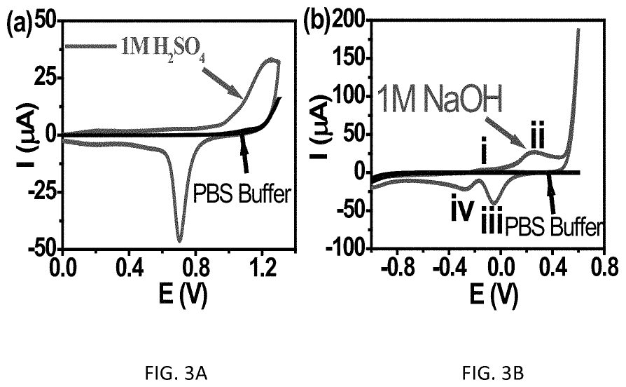

To investigate the electrocatalytic behavior of the AuNPs-modified electrodes, CV was carried out in 1.0 M H₂SO₄ at 50 mV/s. See FIG. 3A. The voltammetric curve of AuNPs/SPE measured in 1.0 M H₂SO₄ showed oxidation in the positive scan (1.2 V) with the formation of AuOH. This is followed by formation of gold oxide monolayers, such as AuO or Au₂O₃. In the reverse scan, a major cathodic peak appears at 0.69 V corresponding to the reduction of gold oxide to metallic gold. (O'Mullane, From Single Crystal Surfaces to Single Atoms: Investigating Active Sites in Electrocatalysis. Nanoscale 2014, 6 (8), 4012-4026.) However, in PBS electrolyte, no distinguishable current response for AuNPs/SPE was observed. The voltammogram of AuNPs/SPE in alkaline medium (1.0 M NaOH) resembled that of a bulk gold electrode. See FIG. 3B. The anodic peak (FIG. 3B, “i”) is because of the anodic discharge of water, with the formation of a sub monolayer of adsorbed hydroxyl radicals. The oxidation wave (FIG. 3B, “ii”) is related to Au(III) formation. The cathodic peaks (“iii” and “iv” in FIG. 3B) in the negative sweep are linked to the reduction of Au(III) species. However, in PBS electrolyte, no distinguishable current response for AuNPs-modified electrode was observed. Anu et al., Synthesis of Imidazole-Based NHC—Au(I) Complexes and Their Application in Non-Enzymatic Glucose Sensing. J Appl Electrochem 2013, 43 (9), 939-951.

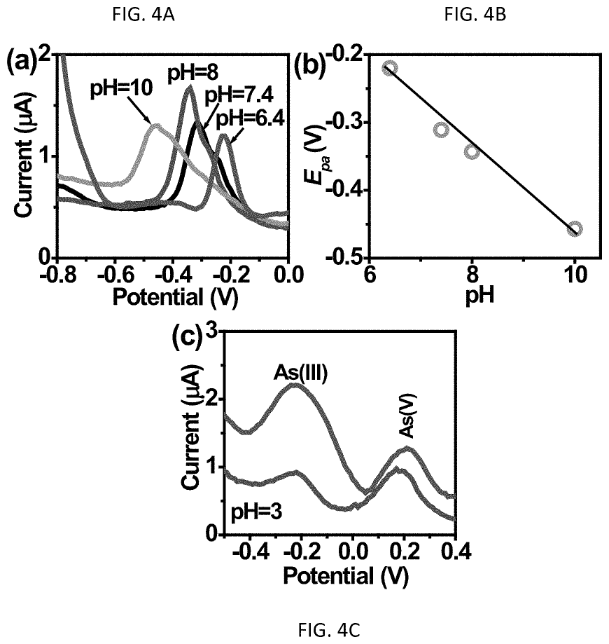

Effect of pH: The voltammetric behavior of metal ions is strongly influenced by the pH of the supporting electrolyte solution. Thus, the behavior of arsenic ions in solutions of various pH was explored. FIG. 4A shows the effect of pH on the separation of peak potentials. The speciation of arsenic is different at various pH values, and thus pH would be expected to influence the precise detection of arsenic. For the toxic As(III) species, the non-ionic arsenic molecule H₃AsO₃ is the dominant molecular form of arsenic at pH below about 7.0. When the pH is significantly greater than 7.0, ionized As(III) species in the form of H₂AsO₃⁻ are created (pH ˜7.0 to ˜8.0). When the pH value reaches pH 10.0, the ionized As(III) species HAsO₃²⁻ are created. The peak potentials shifted negatively with pH of the solutions in the range of about pH 6.4 to pH 10 due to the different arsenic types in different pH medium. Again, see FIG. 4A. A plot of reduction potential vs. pH (Eₚ vs. pH) showed a linear line with a slope value of (−∂Ep/∂pH)˜60 pH⁻¹ which is close to the Nernstian value (FIG. 4B). As(V) is generally considered electrochemically inert under normal conditions, but can be directly electro-reduced to As(0) at pH below 3.5. The electrochemistry of As(III) and As(V) at the AuNPs/SPE was examined at pH 3.0 (FIG. 4C). Given that the As electrochemistry is pH-dependent, two well-resolved anodic peaks, corresponding to As(III) and As(V), were obtained. Thus, using the DPASV mode, it is possible to selectively determine trace amounts of As(III) against As(V). Yang et al., Electrochemical Detection of Trace Arsenic(III) by Nanocomposite of Nanorod-Like α-MnO2 Decorated with ˜5 nm Au Nanoparticles: Considering the Change of Arsenic Speciation. Anal. Chem. 2016, 88 (19), 9720-9728. Xu et al., Enhanced Arsenic Removal from Water by Hierarchically Porous CeO2-ZrO2 Nanospheres: Role of Surface- and Structure-Dependent Properties. Journal of Hazardous Materials 2013, 260, 498-507. Vishnu et al., Selective Electrochemical Polymerization of 1-Napthylamine on Carbon Electrodes and Its PH Sensing Behavior in Non-Invasive Body Fluids Useful in Clinical Applications. Sensors and Actuators B: Chemical 2018, 275, 31-42.

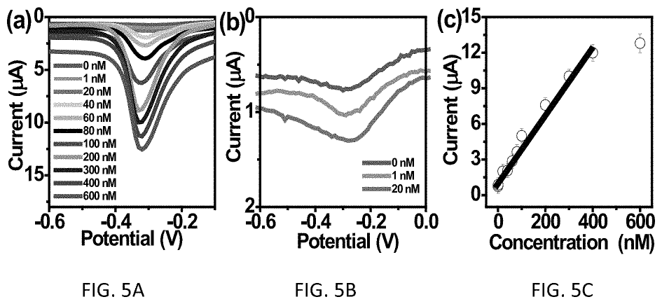

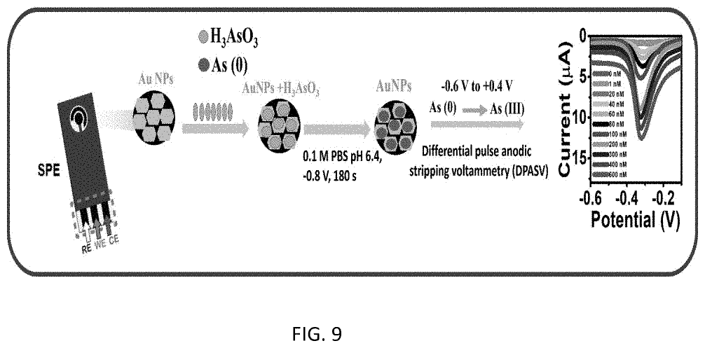

Electrochemical detection of As(III). FIG. 5A shows differential pulse anodic stripping voltammogram (DPASV) for various As(III) concentrations obtained using the AuNPs/SPE described above. The first step in electrochemical detection is the pre-concentration of H₃AsO₃ at the AuNPs/SPE surface from the bulk solution. This is shown schematically in FIG. 9. The second step involves the reduction of arsenic species to As(0) at −0.8 V followed by its stripping (reoxidation of As0 to As(III)). Kempahanumakkagari, S.; Deep, A.; Kim, K.-H.; Kumar Kailasa, S.; Yoon, H.-O. Nanomaterial-Based Electrochemical Sensors for Arsenic—A Review. Biosensors and Bioelectronics 2017, 95, 106-116. Zhou et al., Electrochemically Etched Gold Wire Microelectrode for the Determination of Inorganic Arsenic. Electrochimica Acta 2017, 231, 238-246. Coneo Rodríguez et al., Au Nanoparticles Embedded in Mesoporous ZrO2 Films: Multifunctional Materials for Electrochemical Detection. Sensors and Actuators B: Chemical 2018, 254, 603-612. Dutta et al., Gold Nanostar Electrodes for Heavy Metal Detection. Sensors and Actuators B: Chemical 2019, 281, 383-391. Babu et al., Albumin Capped Carbon-Gold (C—Au) Nanocomposite as an Optical Sensor for the Detection of Arsenic(III). Optical Materials 2018, 84, 339-344. Again, see FIG. 9 which shows this process schematically. The characteristic peak for As(III) was observed at ˜−0.31 V. The predominant arsenic species in water at pH 6.4 is H₃AsO₃. In order to obtain the optimum level of sensitivity for detecting the toxic metal with AuNPs/SPE, the relevant variables (namely: effect of the supporting electrolyte, pH of the supporting electrolyte, the deposition potential (−0.8 V), and the deposition time (180 second)) were optimized. Other parameters such as step potential, amplitude and pulse period were established as 10 mV, 50 mV and 0.5 s, respectively. As shown in FIGS. 5A and 5B, the current peak at −0.31 V increased with increasing concentrations of As(III). The calibration plot for As(III) evaluating the current peak height for each concentration was found to be linear over the range of about 0.075 ppb to about 0.03 ppm (about 1 nM to about 400 nM) with a linear regression equation; I (μA)=1.2534+0.0285 C (nmol L⁻¹) (R²=0.9838) with a sensitivity of 0.0285 μA/nM. The calibration curve is shown in FIG. 5C. The LOQ was about 0.075 ppb (about 1 nM) at 180 seconds deposition time and 60 seconds stripping time. The theoretical LOD was 0.1209 ppb (1.61 nM). Per IUPAC (International Union of Pure and Applied Chemistry) definition, using the standard approach of alternative (SA), LOD_{SA}=3 S/q. Where, S is the standard deviation of the blank signal, and q is the slope of the calibration curve. The LOQ and LOD obtained using the AuNPs/SPE is well below the 10 ppb maximum arsenic concentration promulgated by the U.S. Environmental Protection Agency. The LOQ of the sensor disclosed herein is about 1 nM As. Thus, the AuNPs/SPE is useful for onsite detection and quantitation of trace quantities of As(III) in water. The LOQ of the subject electrode is among the lowest (i.e., best) reported to date.

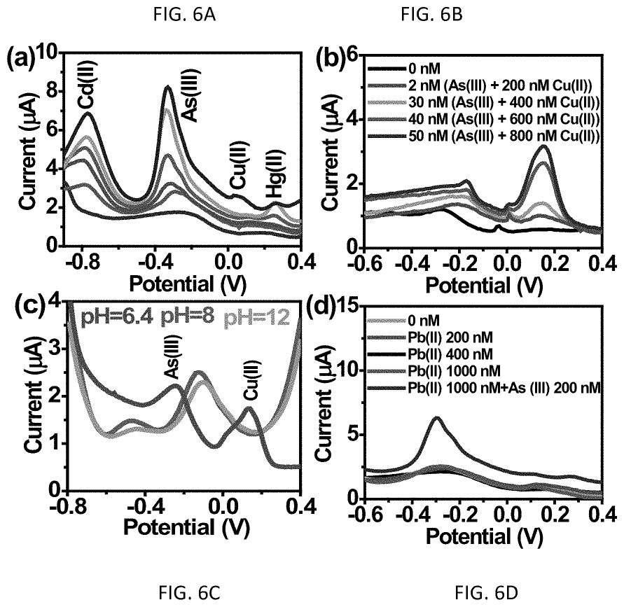

Interference study: The selectivity of the method for arsenic detection was investigated by comparing peak potential toward As(III) versus peak potential toward interfering ions. Reliable detection of trace As(III) in an authentic sample (tap water) without interference from other organic or inorganic ions is a challenging task because, for example, other metal ions present in the authentic samples can be co-precipitated and stripped-off the electrode along with the As(III), thereby confounding the calculation of arsenic concentration. An electrode made as described herein was challenged by spiking test samples with known concentrations of Cu(II), Hg(II), and Cd(II). The concentrations of the potentially interfering species ranged from 0 ppb to 75 ppb (0 nM to 1000 nM) with As(III) ranging from 0 ppb to 15 ppb (0 nM to 200 nM) simultaneously. See 6A. It is well-known that Cu(II) is a major interfering ion in the detection of As(III). The interference of Cu(II) in detecting As(III) arises from the formation of intermetallic compounds such as Cu₃As₂. However, in the interference study the stripping peak potential of As(III) is at −0.31 V, which is well separated from Cu(II). See FIG. 6A. The voltammograms for the binary mixture of As(III) and Cu(II) were also well separated from each other with a potential difference of ΔE_{As(III)-Cu(III)}=305 mV. It was also shown that Cd(II) and Hg(II) likewise exhibit no mutual interference; their respective peaks are also well separated (See FIG. 6A and FIG. 6B). (39) Dai, X.; Nekrassova, O.; Hyde, M. E.; Compton, R. G. Anodic Stripping Voltammetry of Arsenic(III) Using Gold Nanoparticle-Modified Electrodes. Anal. Chem. 2004, 76 (19), 5924-5929. Carrera et al., Electrochemical Determination of Arsenic in Natural Waters Using Carbon Fiber Ultra-Microelectrodes Modified with Gold Nanoparticles. Talanta 2017, 166, 198-206. Yang et al., Electrochemical Determination of Arsenic(III) with Ultra-High Anti-Interference Performance Using Au—Cu Bimetallic Nanoparticles. Sensors and Actuators B: Chemical 2016, 231, 70-78. Ordeig et al., Trace Detection of Mercury(II) Using Gold Ultra-Microelectrode Arrays. Electroanalysis 2006, 18 (6), 573-578.

The influence of pH on the simultaneous measuring of As(III) and Cu(II) concentration was investigated at the AuNPs/SPE. Here, the pH of the supporting electrolyte was varied in a wider range of from pH 6.8 to pH 10. The investigation was conducted via DPASV with test solutions containing 50 nM As(III) and 500 nM Cu(II). The results are shown in FIG. 6C. At pH 6.4, pH 8, and pH 10, the As(III) and Cu(II) peaks are well separated. At acidic pH, the potential shifts towards right and at basic pH peak potential shift towards left. Additionally, the potential interference of Pb(II) with As(III) was also investigated. There was no mutual interference between As(III) and Pb(II). See FIG. 6D. No marked interference or alterations in the current signals were noticed for anions Cl⁻, SO₄²⁻, and NO₃⁻ (1 mM each, figure not shown).

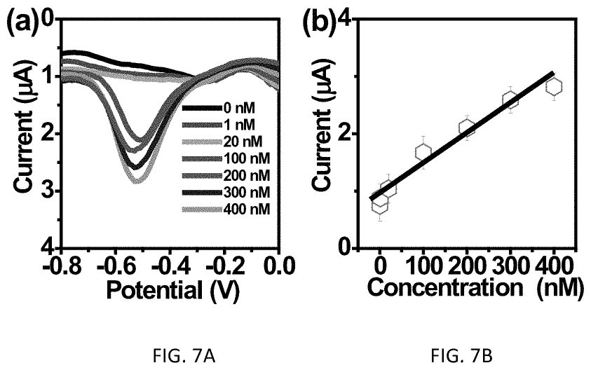

Analytical application: To evaluate the utility of the AuNPs/SPE to detect and measure the concentration of arsenic in drinking water, tap water samples (directly from the spigot; pH 7.8) were tested using the AuNPs/SPE. Tap water is a complex solution containing various metals (Al, Fe, and Mn), organic matter, and other contaminants. In the tap water samples tested, it was seen that there was no Cu, As, Zn, Cr, or Pt. DPASV responses are displayed in FIGS. 7A and 7B. As shown in FIG. 7A, the current peak at −0.51 V increases proportionally with an increase in concentration of As(III) stock solution. The calibration plot for As(III) was found to be linear over the range of 0.075 ppb to 0.03 ppm (1 nM to 400 nM) with a linear regression equation; I (μA)=0.93+0.052 C (nmol L⁻¹) (R²=0.9652), and a sensitivity of 0.052 μA/μM as shown in FIG. 7B. The LOQ was 0.075 ppb (1 nM) at a 180 second deposition time and 60 second stripping time. The theoretical LOD was 0.038 ppb (0.51 nM). As noted above, these LOQ and LOD figures using the subject AuNPs/SPE are well below the 10 ppb upper limit for arsenic in drinking water set by the WHO and the U.S. E.P.A.

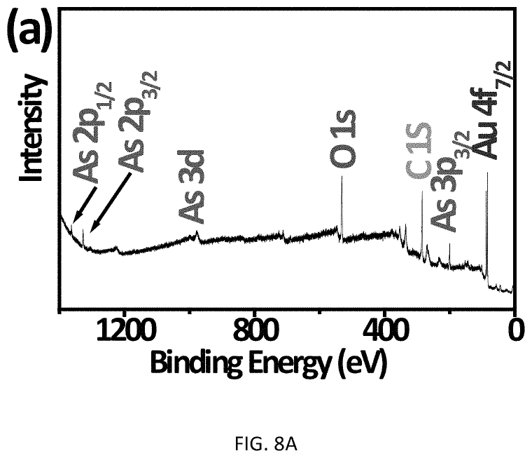

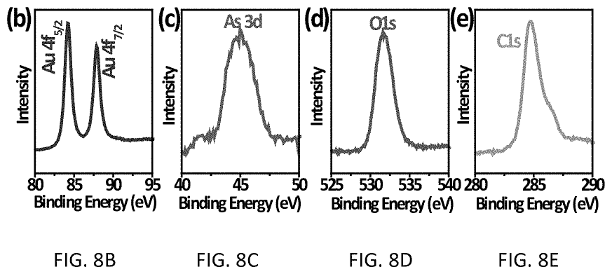

Mechanistic study of As(III) detection at the AuNPs/SPE: Additional studies were conducted to confirm the deposition of As(III) on the AuNPs working surface. The XPS survey spectrum of As(III) detection at AuNPs/SPE electrode reveals multiple regions of Au 4f, As 3d, O 1s, and C 1s respectively. See FIG. 8A. Individual spectra for each element (Au 4f, As 3d, O 1s, and C 1s) accumulated versus binding energy are shown in FIGS. 8B, 8C, 8D, and 8E, respectively. Two unique lines separated by 3.78 eV were observed, specifically the Au 4f_{5/2} and Au 4f_{7/2} lines, which are due to the spin-orbit splitting of the Au 4f level. See FIG. 8B. A clear As 3d peak reveals a binding energy of 44.9 eV, thus confirming the presence of arsenic on the adsorbent. See FIG. 8C. The peaks in the O 1s spectrum at a binding energy of 531.4 eV are due to oxygen in the form of As₂O₃ (FIG. 8D). There is additionally a small shift in O 1s BE upon As(III) interaction with AuNPs/SPE (531.4 eV), in comparison to standard O 1s BE (529.0 eV), supporting strong interaction of O²⁻ with AuNPs. The peak in the C 1s area is from the screen-printed electrode itself (FIG. 8E). On the whole, XPS evaluation validates As₂O₃ at the AuNPs/SPE because the average binding energy values of As₂O₃ (3d_{5/2}) is 45 eV and that of As₂O₅ (3d_{5/2}) is above 46 eV, which clearly indicates that As(III) was detected at the electrode surface (and not As(V)). The intense As peak, present even after washing with copious amounts of buffer, is indicative of a strong interaction between the Au—As(III).

Jia et al., The Electrochemical Reaction Mechanism of Arsenic Deposition on an Au(111) Electrode. Journal of Electroanalytical Chemistry 2006, 587 (2), 247-253. Zhao et al., MOF Derived Iron Oxide-Based Smart Plasmonic Ag/Au Hollow and Porous Nanoshells “Ultra-Microelectrodes” for Ultra-Sensitive Detection of Arsenic. Journal of Materials Chemistry A 2018, 6 (33), 16164-16169. Pourbeyram and Asadi, Time Resolved Direct Determination of Arsenate in the Presence of Arsenite on Pencil Graphite Electrode Modified by Graphene Oxide and Zirconium. Electroanalysis 2018, 30 (1), 154-161.

Repeatability, reproducibility and stability: Duplicate assays were also performed to test repeatability. The repeatability of AuNPs/SPE to detect As(III) in terms of relative standard deviations for 200 nM of As(III) was less than 1.1% (n=3). The reusability of the AuNPs/SPE was also estimated by testing the response to 200 nM As(III) after storing at 25° C. for each 2 days and over 6 days. The sensor maintained at least 99% of the initial current in the continuous test. The storage stability of the modified electrode was measured after two weeks storage. After the storage, the DPASV response of the As(III) exhibited 97.2% of the initial value. It indicates that the AuNPs/SPE shows good repeatability, reproducibility and stability toward repetitive deposition-stripping under the optimized condition.

Conclusions: A facile and inexpensive approach for scalable synthesis of colloidal AuNPs is described. Further, the disclosure establishes that AuNPs are useful as an electrocatalyst for the electrochemical detection of As(III). The LOQ and LOD obtained using AuNPs/SPE was 0.075 ppb (1 nM) and 0.1209 ppb (1.61 nM) respectively. AuNPs/SPE allows a detection concentration of As(III) as low as 1 nM. AuNPs/SPE has been successfully applied for the detection of As(III) in tap water. The linear range of detection is from about 0.075 ppb to about 0.03 ppm. The result of speciation about As(III) and As(V) with known concentration was also demonstrated at the AuNPs/SPE. It is interesting to note that the same electrode can be successfully used for the detection of different analytes under the same experimental condition without medium exchange or activation. The AuNPs/SPE showed individual, well-defined voltammetric peaks for As(III), Cu(II), Cd(II), and Hg(II). The analytical application of the AuNPs/SPE for detecting and quantifying As(III) in field samples collected from faucet water in Madison, Wis., was proven.

What is claimed is:

1. A method of detecting arsenic in a sample, the method comprising;
contacting the sample with a working electrode and a counter electrode in the presence of an electrolyte;
wherein the working electrode comprises a nanoparticulate noble metal having a particle size of from about 10 nm to about 20 nm adhered to a supporting electrode material and wherein the working electrode is dimensioned and configured to generate an electrochemical response proportional to concentration of arsenic in the sample; and
applying a potential across the electrodes and determining the electrochemical response of the working electrode to the sample;
wherein the sample is suspected of containing arsenic.

2. The method of claim 1, wherein the working electrode comprises nanoparticulate gold.

3. The method of claim 2, wherein mean particle size of the nanoparticulate gold ranges from about 12 nm to about 18 nm.

4. The method of claim 1, wherein the sample is a liquid.

5. The method of claim 1, wherein the sample is an aqueous sample.

6. The method of claim 1, wherein the potential is applied relative to a reference electrode.

7. The method of claim 6, wherein the working electrode comprises nanoparticulate gold.

8. The method of claim 7, wherein mean particle size of the nanoparticulate gold ranges from about 12 nm to about 18 nm.

9. The method of claim 6, wherein the sample is a liquid.

10. The method of claim 6, wherein the sample is an aqueous sample.

11. The method of claim 6, comprising varying the potential applied relative to the reference electrode and further comprising determining a voltammetric response to the potential applied.

12. The method of claim 11, wherein the working electrode comprises nanoparticulate gold.

13. The method of claim 12, wherein mean particle size of the nanoparticulate gold ranges from about 12 nm to about 18 nm.

14. The method of claim 11, wherein the sample is a liquid.

15. The method of claim 11, wherein the sample is an aqueous sample.

16. A method of detecting arsenic in a sample, the method comprising;
contacting an aqueous sample with a working electrode and a counter electrode in the presence of an electrolyte;
wherein the working electrode comprises a nanoparticulate gold having a particle size of from about 12 nm to about 18 nm adhered to a supporting electrode material and wherein the working electrode is dimensioned and configured to generate an electrochemical response proportional to concentration of arsenic in the sample; and
applying a varying potential across the working electrode and the counter electrode relative to a reference electrode; and
determining a voltammetric response of the working electrode to the potential applied, wherein the voltammetric response is proportional to concentration of arsenic in the aqueous sample.

17. An electrochemical detector comprising, in combination:
a reference electrode;
a counter electrode; and
a working electrode comprising noble metal nanoparticles having a mean particle size of from about 10 nm to about 20 nm, adhered to a supporting electrode material.

18. The electrochemical detector of claim 17, wherein
the reference electrode comprises silver and silver chloride;
the counter electrode comprises carbon; and
the supporting electrode material of the working electrode comprises carbon.

19. The electrochemical detector of claim 17, wherein the reference electrode, the counter electrode, and the working electrode are screen-printed electrodes.

20. The electrochemical detector of claim 17, wherein the noble metal nanoparticles comprise gold nanoparticles.

21. An electrochemical detector made by a process comprising
screen-printing onto a substrate a reference electrode, a counter electrode, and a working electrode; and
drop casting noble metal nanoparticles onto the working electrode.

22. The electrochemical detector of claim 21, comprising drop casting noble metal nanoparticles having a particle size of from 10 nm to 20 nm onto the working electrode.

23. The electrochemical detector of claim 22, comprising drop casting noble metal nanoparticles having a particle size of from 12 nm to 18 nm onto the working electrode.

24. The electrochemical detector of claim 21, wherein
the reference electrode comprises silver and silver chloride;
the counter electrode comprises carbon; and
the working electrode comprises carbon.

25. The electrochemical detector of claim 21, comprising drop casting gold nanoparticles onto the working electrode.
